(19) United States
(12) Patent Application Publication
(10) Pub. No.: US 2007/0292503 A1
(43) Pub. Date: Dec. 20, 2007

(54) ORAL PHARMACEUTICAL COMPOSITION OF POORLY WATER-SOLUBLE ACTIVE SUBSTANCE

(76) Inventor: Henricus R.M. Gorissen, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/762,515

(22) Filed: Jun. 13, 2007

Related U.S. Application Data
(60) Provisional application No. 60/814,076, filed on Jun. 16, 2006.

(51) Int. Cl. A61K 9/20; A61K 31/55

(52) U.S. Cl. 424/464; 514/212.07

(57) ABSTRACT

An oral composition of Benzazepin-1-acetic acid derivatives is provided comprising: a) an active compound in an amount of between 10 and 65% of the total weight of the formulation; b) at least 10% w/w an alkaline compound or a mixture of alkaline compounds; c) optionally comprising auxiliary materials an amount of between 1% and 45% of the total weight of the formulation The invention also provides an oral composition comprising sodium carbonate with a specific particle size and/or surface area as alkaline compound.

This application claims the priority of U.S. provisional application No. 60/814,076, filed Jun. 16, 2006, the entire content of which is incorporated herein by reference.

The present invention relates to an oral formulation of an active compound of the general formula (I):

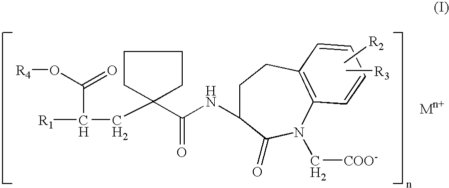

wherein:
R₁ is chosen from:
(1) (C1-C6)alkoxy(C1-C6)alkyl, which is optionally substituted by a (C1-C6)alkoxy;
(2) phenyl-(C1-C6)-alkyl and phenyloxy-(C1-C6)-alkyl, wherein the phenyl group is optionally substituted with (C1-C6)alkyl, (C1-C6)alkoxy or halogen; and
(3) naphtyl-(C1-C6)-alkyl;
R₂ and R₃ are both independently hydrogen or halogen;
R₄ is a biolabile ester forming group;
M is a hydrogen or a metal ion, for example a bivalent metal ion, and
n is 1, 2 or 3.

These compounds and their salts and biolabile esters are embodiments of the present invention and can be potent ECE/NEP inhibitors, which are described in Waldeck et al., U.S. Pat. No. 5,677,297 and EP 0733642. The benzazepine-N-acetic acid compounds used in the present invention are known from EP 0733642, EP 0830863, WO 00/48601 and WO 01/03699, and can be produced by the methods described in U.S. Pat. No. 5,677,297 and EP 0733642. These patents are related to said compounds and their physiologically acceptable salts and to the use of the compounds in heart insufficiency. WO 03/059939 relates to specific salts of these compounds, especially to the calcium salt form. EP 0830863, WO00/48601 and WO01/03699 are related to the use of the above compounds in the improvement of gastrointestinal blood flow, in the treatment of hypertension, and in the treatment and prophylaxis of cardiac damages induced by adriamycin and comparable anti-cancer drugs, respectively.

Various active compounds, including the compounds of formula (I) mentioned above have very poor solubility in water. When these active compounds are administered to the body, they often have poor bio-availability due to their poor solubility in the digestive fluid. In order to solve this problem, several methods were developed, such as micronization, inclusion in cyclodextrins, the use of inert water-soluble carriers, the use of solid dispersions (WO 00/00179) or solid solutions or nanocrystalline or amorphous forms of the active compound.

WO 03/068266 describes an oral solid solution formulation of compounds of formula (I) having enhanced bio-availability compared with said active compound in a traditionally formulated form. Although this formulation has good bioavailability properties, it has the draw-back of being formed via a melt mixture, which leads to some restrictions, such as the need to formulate either into a capsule or into a tablet via melt-extrusion technique. Furthermore, the size of the formulation will be too large for higher dosages.

WO 2006/067150 describes an oral immediate release formulation of compounds of formula (I) comprising up to 60% of the total weight of the formulation comprising the active compound, at least 10 % w/w of an alkaline compound or a mixture of alkaline compounds, between 0.1 and 10% w/w of one or more surfactants and, optionally, auxiliary materials in an amount of between 1% and 45% of the total weight of the formulation. When docusate sodium is used as the surfactant, there is good bioavailability of the active compound.

The present invention provides an alternative oral formulation for the compound of formula I, as defined above, with significantly increased bio-availability compared with said active compound in a traditionally formulated form. The oral formulation of the invention is sufficiently stable for commercial use and also can be used to prepare formulations with a high content of active compound with a reasonable size. Furthermore, the oral formulation of the invention can be prepared using normal formulation procedures and equipment, so that large investments are not necessary.

The invention relates to an oral formulation of an active compound of the general formula (I):

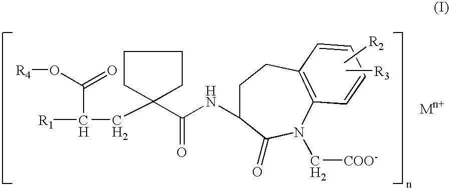

wherein:
R₁ is chosen from:
(1) (C1-C6)alkoxy(C1-C6)alkyl, which is optionally substituted by a (C1-C6)alkoxy:
(2) phenyl-(C1-C6)-alkyl and phenyloxy-(C1-C6)-alkyl, wherein the phenyl group may be substituted with (C1-C6)alkyl, (C1-C6)alkoxy or halogen, and
(3) naphtyl-(C1-C6)-alkyl;
R₂ and R₃ are both independently hydrogen or halogen;
R₄ is a biolabile ester forming group;
   M is a hydrogen or a metal ions preferably a bivalent metal ion; and
   n is 1, 2 or 3, comprising
a) said active compound in an amount ranging between 10% and 80% of the total weight of the formulation;
b) at least 10% w/w of an alkaline compound or a mixture of alkaline compounds;
c) optionally comprising at least one auxiliary material in an amount ranging between 1% and 45% of the total weight of the formulation; and wherein
the formulation does not contain a surfactant.

M can be chosen from Li+, Ca2+, Mg2+ and Zn2+, and can be Ca2+. (C1-C6)-alkyl is defined as a straight or branched alkyl group comprising between 1 and 6 carbon atoms. (C1-C6)-alkoxy is defined as a straight or branched alkoxy group comprising between 1 and 6 carbon atoms. R₁ can be phenylethyl, R₂ and R₃ can be hydrogen, and R₄ can be ethyl.

In the framework of the present invention, suitable R₄ groups that can form biolabile esters include lower alkyl groups, phenyl or phenyl-lower-alkyl groups, which can be optionally substituted in the phenyl ring by lower alkyl or by a lower alkylene chain bonded to two adjacent carbon atoms, dioxolanylmethyl groups, which can be optionally substituted in the dioxolane ring by lower alkyl, or C2-C6-alkanoyloxymethyl groups, which are optionally substituted on the oxymethyl group by lower alkyl. Where the group R₄ forming a biolabile ester is lower alkyl, it can be an unbranched alkyl group with 1 to 4, for example 2, carbon atoms. Where the group forming a biolabile ester can be an optionally substituted phenyl-lower-alkyl group, its alkylene chain can contain 1 to 3, for example 1, carbon atoms. Where the phenyl ring is substituted by a lower alkylene chain, it can contain 3 to 4, for example 3, carbon atoms. Suitable phenyl-containing substituents R₄ are phenyl, benzyl and indanyl. Where R₄ is an optionally substituted alkanoyloxymethyl group, its alkanoyloxy group can contain 2 to 6, for example 3 to 5, carbon atoms and can be branched and can be, for example, a pivaloyloxymethyl radical (tert-butylcarbonyloxymethyl radical).

An example of a compound for use in the invention is the calcium salt of the acid, 3-[[[1-[2-(ethoxycarbonyl)-4-phenylbutyl]cyclopentyl]carbonyl]amino]-2,3,4,5-tetrahydro-2-oxo-1H-1-benzazepi-ne-1-acetic. Another example of compound according to the invention is its 3S,2′R form, also known as daglutril calcium or SLV 306 Calcium. This compound is referred to as Compound S—Ca, the corresponding acid (3-[[[1-[2-(ethoxycarbonyl)-4-phenyl-butyl]cyclopentyl]-carbonyl]amino]-2,3,4,5-tetrahydro-2-oxo-1H-1-Benzazepine-1-acetic acid), also known as daglutril or SLV306 is referred to as Compound S—H.

The active compound of formula (I), can be used in an amount between about 10 and 80% by weight, for example in an amount ranging between 15 and 75% by weight, and, as another example in an amount ranging between 20 and 65% by weight and, as a further example, in an amount ranging between about 45 and 65% by weight. The active compound can be or may optionally be used in a micronized form.

The following definitions are provided to facilitate understanding of certain terms used within the framework of the present application.

“Sufficiently stable for commercial use” means acceptable chemical and physical stability during a storage period of at least one year at ambient conditions, for example at least 2 years, or for at least 3 years and even for at least 5 years. “Acceptable chemical stability” means not more than 5% degradation of the active material during the storage period, for example not more than 3%, and as a further example, not more than 1%. “Acceptable physical stability” means there is no significant change in appearance, no breaking of tablet during deblistering at the end of the storage period, and not more than 20% change of the disintegration time. The term “micronized” refers to a particle size wherein, on a volume basis, more than 95% of the particles is smaller than 75 microns.

“Surfactants” are defined as molecules with well defined polar and non-polar regions that allow them to aggregate in solutions to form micelles. Depending on the nature of the polar area, surfactants can be non-ionic, anionic, cationic, and zwitterionic. Examples of non-ionic hydrophilic surfactants are polyoxyethylene sorbitan esters, cremophores, and poloxamers. Examples of anionic surfactants are sodium lauryl sarcosinate, docusate, and pharmaceutically acceptable docusate salts, such as docusate calcium, docusate sodium, and docusate potassium.

The alkaline compound is chosen from inorganic and organic alkaline compounds, such as sodium bicarbonate, potassium bicarbonate, sodium carbonate, potassium carbonate, sodium citrate, tris buffer, triethanolamine, alkaline hydroxides, such as sodium hydroxide, potassium hydroxide or magnesium hydroxide, and alkaline phosphates, such as dipotassium hydrogen phosphate, and meglumine. Mixtures of these alkaline compounds can also be used. Alkaline compounds that can be used include sodium bicarbonate, potassium bicarbonate, sodium carbonate, potassium carbonate, and calcium carbonate A specific example of an alkaline compound is sodium carbonate.

The alkali system can be present in an amount greater than 10% w/w of the uncoated composition, such as greater than 20% w/w, or is present in an amount greater than 25%, 30% w/w, 40% w/w, 45% w/w, 50% w/w, 55% w/w or 60% w/w of the uncoated composition. The term “uncoated formulation” means a formulation before the application of the optional coating material(s).

If a carbonate is used, it can be used in an amount of 25% of the total weight of the uncoated formulation or it is present in an amount of at least 45% w/w, or is present in an amount greater than 50% w/w, 55% w/w or 60% w/w of the uncoated formulation.

Another embodiment of the invention relates to an oral pharmaceutical composition of an active compound of the general formula (I):

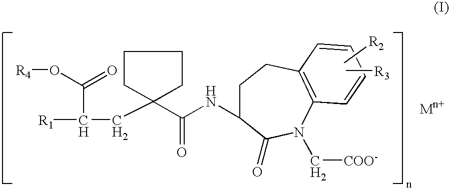

wherein:
R₁ is chosen from
(1)(C1-C6)alkoxy(C1-C6)alkyl, which is optionally substituted by a (C1-C6)alkoxy;
(2) phenyl-(C1-C6)-alkyl and phenyloxy-(C1-C6)-alkyl, wherein the phenyl group is optionally substituted with (C1-C6)alkyl, (C1-C6)alkoxy or halogen; and
(3) naphtyl-(C1-C6)-alkyl;
R₂ and R₃ are both independently hydrogen or halogen;
R₄ is a biolabile ester forming group;
M is a hydrogen or a metal ion, including a bivalent metal ion; and
n is 1, 2 or 3;
comprising
a) said active compound in an amount ranging from 10 to 65% of the total weight of the formulation;
b) at least 10% w/w of a sodium carbonate having a particle size distribution, wherein more than 97% of the particles are smaller than approximately 500 μm, more than 40% of the particles are smaller than approximately 160 μm, and more than 10% of the particles are smaller than approximately 63 μm; and
c) optionally comprises at least one auxiliary material in an amount ranging between 1% and 45% of the total weight of the formulation.

In one embodiment, the alkali system comprises a sodium carbonate having a smaller particle size than normal sodium carbonate, which has a particle size distribution (determined by sieve analysis and based on weight) wherein at most 25% of the particles are smaller than approximately 160 μm. This sodium carbonate also has higher specific surface area than normal sodium carbonate, which has a specific surface area (determined according to the standard BET area measurement) of approximately 0.2 m²/g. As indicated above, the sodium carbonate used in the embodiment has a particle size distribution (determined by sieve analysis and based on weight) wherein more than 97% of the particles are smaller than approximately 500 μm, more than 40% of the particles are smaller than approximately 160 μm, and more than 10% of the particles are smaller than approximately 63 μm. In a another embodiment more than 98% of the particles are smaller than approximately 500 μm, more than 60% of the particles are smaller than approximately 160 μm and more than 30% of the particles are smaller than approximately 63 μm. The specific surface area can be higher than 1 m²/g, and also higher than 1.5 m²/g. An exemplary sodium carbonate is sodium carbonate marketed by Solvay SA as Soda Ash IPH. In this type of sodium carbonate, typically 99.8% of the particles are smaller than approximately 500 μm, 80% of the particles are smaller than approximately 160 μm, and 40% of the particles are smaller than approximately 63 μm. This type of sodium carbonate has a specific surface area of 2 m²/g.

Unexpectedly, use of an alkaline compound in the formulation, alone or in a mixture, even in the absence of surfactant, prevents the the formation of a difficult to solubilize gel in acid gastric fluid, thereby enhancing the solubility of SLV-306. In vitro dissolution studies demonstrate the prevention of gel formation in a biphasic dissolution model (see Example la), which indicates an improvement in in vivo solubility, as well as an improvement in bioavailability. Use of sodium carbonate with a mean particle size of 100 μm and a specific surface area of 2 m²/g such as (Soda Ash IPH) provides a formulation with good in vivo solubility and good bioavailability. Further the compositions are expected to have good stability upon storage.

If the abovementioned carbonate with specific mean particle size and surface area is used, it is can be used in an amount of at least 15% of the total weight of the uncoated formulation, or in an amount of at least 18%, as well as in an amount of at least 20%, or it is present in an amount greater than 25% w/w, 30% w/w, 40% w/w, 50% w/w or 60% w/w of the uncoated formulation.

Specific solid alkaline compounds like the bicarbonates and carbonates as indicated above are often used in combination with solid acidic compounds (including, but not limited to, citric acid, tartaric acid, adipic acid, fumaric acid, succinic acid, ascorbic acid, nicotinic acid, saccharin, aspirin, malic acid, sodium dihydrogen phosphate, disodium dihydrogen pyrophosphate, sodium dihydrogen citrate, and disodium hydrogen citrate) in effervescent compositions. In some embodiments, the composition does not contain an acidic compound.

The formulation optionally comprises auxiliary materials in an amount of up to 45% of the total weight of the formulation and possibly between 1 % and 45% of the total weight of the formulation. Examples of these auxiliary materials include, but are not limited to:

Binders including, but not limited to, acacia, alginic acid and salts thereof, cellulose derivatives, methylcellulose, hydroxyethyl cellulose, hydroxypropyl cellulose, polyethylene glycol, gums, polysaccharide acids, hydroxypropyl methylcellulose, gelatin, polyvinylpyrrolidone, polyvinylpyrrolidone/vinyl acetate copolymer, polymethacrylates, hydroxypropylmethylcellulose, starch, pregelatinized starch, ethylcellulose, tragacanth, dextrin, microcrystalline cellulose, sucrose, and glucose.

Disintegration agents, including but not limited to starches, pregelatinized corn starch, pregelatinized starch, celluloses, cross-linked carboxymethylcellulose, crospovidone, cross-linked polyvinylpyrrolidone, a calcium or a sodium alginate complex, clays, alginates, gums, or sodium starch glycolate, and any disintegration agents used in tablet preparations.

Filling agents, including, but not limited to, lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, and sorbitol.

Stabilizers, including, but not limited to, any antioxidation agents, buffers, or acids.

Lubricants, including, but not limited to magnesium stearate, calcium hydroxide, talc, colloidal silicon dioxide, sodium stearyl fumarate, hydrogenated vegetable oil, stearic acid, glyceryl behenate, magnesium, calcium and sodium stearates, waxes, Stearowet, boric acid, sodium benzoate, sodium acetate, DL-leucine, polyethylene glycols, sodium oleate, and sodium lauryl sulfate.

Wetting agents, including, but not limited to, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, and sodium lauryl sulfate.

Diluents, including, but not limited to, lactose, starch, mannitol, sorbitol, dextrose, microcrystalline cellulose, dibasic calcium phosphate, sucrose-based diluents, confectioners sugar, monobasic calcium sulfate monohydrate, calcium sulfate dihydrate, calcium lactate trihydrate, dextrates, inositol, hydrolyzed cereal solids, amylose, powdered cellulose, calcium carbonate, glycine, and bentonite.

Anti-adherents or glidants, including, but not limited to, colloidal silica, talc, corn starch, DL-leucine, sodium lauryl sulfate, and magnesium, calcium, and sodium stearates.

Pharmaceutically compatible carriers, including, but not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, and pregelatinized starch.

When the formulation contains at least 10% w/w of the sodium carbonate having a specific particle size distribution and/or surface area as described above, the formulation may also contain a surfactant as auxiliary material.

The final formulation can be in the form of granules, compressed tablets, or capsules.

The formulation described above can be prepared using conventional formulation procedures and equipment. In another embodiment of the invention, a method of preparing a formulation as described above comprises the following steps:

Mixing the active compound of formula I with one or more alkaline compounds and, optionally, with one or more auxiliary materials;

Compacting the mixture;

Milling and sieving the granules obtained from the compacting and, optionally, mixing the granules with one or more auxiliary materials; and

Optionally, compressing the mixture into tablets, optionally, followed by coating and/or optionally filling the mixture into capsules.

In another embodiment of the invention, the formulation is prepared with an organic granulation method comprising the following steps:
a) Mixing the active compound with the one or more auxiliary materials;
b) Granulating the mixture with an organic solvent;
c) Removing the organic solvent to obtain granules;
d) Milling and sieving the granules and mixing of the sieved granules with the remaining portion of the auxiliary materials;
e) Optionally, compressing the mixture into tablets, optionally, followed by coating, and/or optionally filling the mixture into capsules.

In the organic granulation method several organic solvents can be used. Examples include, but are not limited to, methyl t-butyl ether (MTBE), dichloromethane, and ethyl acetate.

When the formulations of the present invention are provided in the form of tablets, these tablets can have disintegration times of between 5 minutes and 90 minutes. The disintegration times can be below 60 minutes and can be below 45 minutes. Formulations with short disintegration times can be prepared by using a porous sodium carbonate as available in Soda Ash IPH.

Various additional steps can also be part of the process, such as drying, breaking, sieving, mixing, and packaging.

Another aspect of the invention provides a composition having a favorable release profile. An oral pharmaceutical composition as described above, can have a dissolution of at least 50% within 5 minutes, as measured using the USP apparatus 2 configuration at a paddle speed of 50 rpm at 37.0° C. and at pH 6.8. The dissolution after 5 minutes is possibly at least 55% and can be 60%. After 15 minutes the dissolution is at least 65%, and can be at least 70%, or even 75%. After 30 minutes the dissolution is at least 75%, and can be at least 80% or even at least 85%.

At a pH of 2.0 the pharmaceutical composition according to the present invention does not significantly release the active compound. Less than 5% of the active compound is released within 30 minutes, as measured using the USP apparatus 2 configuration at a paddle speed of 50 rpm at 37.0° C. and at pH 2.0. Less than 2% is released and possibly less than 1%.

The following examples are intended to further illustrate the invention, in more detail, and therefore these example are not deemed to restrict the scope of the invention in any way.

### BRIEF DESCRIPTION OF THE DRAWING

FIG. 1 depicts the release profile of the formulations provided in the Examples, wherein ◯=Formulation F; □=Formulation G; ⋄=Formulation H; Δ=Formulation I; ▪=Formulation J; ●=Formulation K.

### EXAMPLES

### Example I

### Materials, Equipment and Methods

Materials

S—Ca was prepared according to the prescription given in Examples 2 and 3 of WO03/059939 starting with the acid prepared according to Example 2 of EP 0733642.

Soda Ash IPH (also indicated as Soda, porous or porous soda) was obtained from Solvay SA Brussels, Belgium.

All other auxiliary materials are readily commercially available.

Equipment

For roller compaction a Fitzpatrick type IR200 roller compactor was used, equipped with a Fitzmill L1A.

The settings of the roller compaction were:
Rotational speed horizontal feeding screw (HFS in rpm)
Rotational speed vertical feeding screw (VFS in rpm)
Rotational speed rollers (N1 in rpm)
Gap (d in mm)
Compaction force (F in KN/cm)
The settings of the Fitzmill were:
Rotational speed hammer knifes (N2 in rpm)
Screen size (in mm)
   Methods

Description of the bi-phase in-vitro dissolution method

The bi-phase dissolution was performed with the USP apparatus 2 configuration. The paddle speed was 50 rpm and the temperature of the vessels (and so the dissolution medium) was maintained at 37.0° C. using Vankel VK7010 equipment.

The dissolution of the formulations was started in 500 ml 0.1 M hydrochloric acid (4.2 ml concentrated hydrochloric acid (HCl) in 500 ml water) (phase 1). After 0, 5, 15, and 30 minutes a sample was taken. After 30 minutes 500 ml 1 M phosphate buffer (32.4 gram sodium di-hydrogen phosphate NaH2PO4 and 124.8 gram di-sodium hydrogen phosphate (Na2HPO4) in 1000 ml water was added to phase 1. Addition of the phosphate buffer changed the pH of the dissolution medium from pH 1 in phase 1 to pH 6.8 in phase 2. During the dissolution test the pH of both phases remained unchanged. Samples were taken after 35, 45, and 60 minutes.

All the samples were filtered through a Pall Zymark Acrodisc PSF, GxF/GHP, 0.45 μm or a Millipore Millex-FH (hydrophobic PTFE 0.45 μm) filter.

The quantity of the dissolved daglutril in the filtered samples was analyzed by off-line UV measurements at 240 nm using external standardization.

In an earlier comparative study with the calcium salt of the compound SLV306 (S—Ca) it was shown that this bi-phase in vitro dissolution method had a good correlation with in-vivo results.

Description of Methods to Characterize Porous Sodium Carbonate

The particle size distribution of the porous sodium carbonate was measured by means of mechanical system. A sample of about 70 g of the product was weighed and placed into the upper sieve of a sifting machine (an automatic device which can transfer a combination of horizontal movements and vertical jerk movements to a set of sieves, e.g. a ROTAP or AS 200 RETSCH sifting machine) containing sieves with screens of 0.5, 0.25, 0.16, 0.125, 0.1, 0.063, and 0.045 mm. The sieving procedure was performed for about 15 minutes. The content of each sieve was weighed and the mass of the particles having a particle size less than 500 μm, the mass of the particles having a particle size less than 160 μm, and the mass of the particles having a particles size less than 63 μm were calculated. The specific surface area of the porous sodium carbonate was measured according to the standard BET area method.

Description of Other Physical Methods

The powder flow was measured in a brass funnel with Ø 8 mm outlet. Powder flow was expressed in sec/100 gram.

The particle size distribution of roller-compacted material was obtained from manual sifting using 0.25 mm, 0.50 mm, 0.71 mm, 0.85 mm 1.0 mm, and 2.0 mm screens. The amount (%)<0.25 mm, the amount (%)>1.0 mm and d(50%) were calculated.

The bulk and tapped density of a mixed granulate was determined as follows:

An amount of 100 to 150 gram granulate was filled in a graduated cylinder.

The occupied volume was determined.

After 1200 taps, the occupied volume was determined again. The Carr-index was calculated from bulk and tapped volume.

Crushing strength of the tablets was determined by crushing five tablets in a Schleuniger hardness tester. The mean value was reported.

Friability was determined on 20 tablets using an Erweka friability tester. The test conditions were 10 minutes at 40 rpm. The friability was expressed in % tablet weight loss.

Disintegration was tested on one tablet, using water as the dissolution medium.

### Example 2

### Preparation of a Traditionally Formulated Coated Tablet of S—Ca

| | Quantity (mg/tablet) |
|---|---|
| Ingredients | Tablet 400/700 mg |
| S—Ca | 414.25 |
| Micro crystalline cellulose PH301 | 249.00 |
| Cross-linked polyvinylpyrrolidon | 14.00 |
| Sodium stearyl fumarate | 1.75 |
| Opadry II Yellow coating | 21.00 |
| Tablet mass | 700.00 |

Procedure

(i) S—Ca was compacted and passed through a 1.0 mm sieve.

(ii) The material of step (i) was mixed with micro crystalline cellulose PH301, cross-linked polyvinylpyrrolidon and sodium stearyl fumarate to obtain a uniform mixture.

(iii) The material of step (ii) was compressed using a tablet compression machine.

(iv) The tablets from step (iii) were coated in suitable coating equipment.

### Example 3

### Preparation of Uncoated Tablets of S—Ca Containing Soda Ash IPH

1. Roller compaction of part of the ingredients

| | Batch granulate obtained | | | | |
|---|---|---|---|---|---|
| | A | B | C | D | E |
| S—Ca | 622 | 622 | 622 | 622 | 622 |
| Soda, porous | 188.6 | 315.5 | 188.6 | 188.6 | — |
| MCC | — | — | — | — | — |
| Lactose 200 m | — | — | — | — | 126.9 |
| Primojel | — | 20 | 20 | 20 | 20 |
| Mg stearate | 2.5 | 2.5 | 2.5 | 2.5 | 2.5 |
| Compaction process | | | | | |
| HFS (rpm) | 37 | 50 | 50 | 50 | 30 |
| VFS (rpm) | 225 | 225 | 225 | 225 | 225 |
| N1 (rpm) | 3 | 3 | 3 | 3 | 3 |
| Gap (mm) | 0.6 | 0.6 | 0.5 | 0.7 | 0.7 |
| F (KN/cm) | 5 | 5 | 5 | 5 | 3 |
| Mill process | | | | | |
| N2 (rpm) | 500 | 500 | 500 | 500 | 500 |
| Screen (mm) | 2.5 | 2.5 | 2.5 | 2.5 | 2.5 |
| Powder flow (sec/100 g) | 38 | 36 | 33 | 34 | 40 |
| % <0.25 mm | 22 | 22 | 19 | 16 | 21 |
| % >1.0 mm | 37 | 42 | 44 | 48 | 39 |
| d(50%) in microns | 720 | 800 | 820 | 950 | 750 |

Procedure:

(i) S—Ca, Soda Ash IPH, Magnesium stearate and Sodium starch glycolate (primojel) were sifted through #40 mesh sieve.

(ii) S—Ca, Soda Ash IPH (indicated as internal) and a portion (indicated as internal) of Magnesium stearate and optionally the Sodium starch glycolate (Primojel®)(indicated as internal) and/or lactose sifted above were mixed to obtain a uniform mixture.

(iii) The material of step (ii) was mixed with a roller compactor at the indicated settings.

(iv) The mixed material was milled and sieved over a screen size of 2.5 mm.

(v) The powder flow and particle size distribution were measured.

2. Tablet Formulation

| | Batch tablet | | | | | | | | | |
|---|---|---|---|---|---|---|---|---|---|---|
| | F | | G | | H | | I | | J | |
| | Batch granulate | | | | | | | | | |
| | A | | B | | C | | D | | E | |
| Composition tablet | 622 | | 622 | | 622 | | 622 | | 622 | |
| (mg/t) | | | | | | | | | | |
| Daglutril calcium (S—Ca) | 188.6 | | 315.5 | | 188.6 | | 188.6 | | — | |
| Soda ash IPH; internal | — | | — | | — | | 126.9 | | 188.6 | |
| Soda ash IPH; external | 126.9 | | — | | 126.9 | | — | | — | |
| MCC PH200; external | — | | — | | — | | — | | 126.9 | |
| Lactose 200 m; internal | — | | 20 | | 20 | | 20 | | 20 | |
| Primojel; internal | 20 | | — | | — | | — | | — | |
| Primojel; external | 2.5 | | 2.5 | | 2.5 | | 2.5 | | 2.5 | |
| Mg stearate; internal | 5.0 | | 5.0 | | 5.0 | | 5.0 | | 5.0 | |
| Mg stearate; external | | | | | | | | | | |
| Total non coated tablet | 965 | | 965 | | 965 | | 965 | | 965 | |
| mass | | | | | | | | | | |
| Bulk/tapped density | 0.55/ | | 0.58/ | | — | | 0.64/ | | 0.61/ | |
| (g/ml) | 0.67 | | 0.70 | | | | 0.76 | | 0.74 | |
| Carr-Index (%) | 17.9 | | 17.1 | | — | | 15.8 | | 17.6 | |
| Compression force | 9 | 14 | 12 | 25 | 6 | 15 | 6 | 11 | 5 | 10 |
| (KN) | | | | | | | | | | |
| Tablet weight | 836 | — | 901 | — | 883 | — | — | 977 | — | 893 |
| Friability (%) | 0.3 | — | 0.7 | — | 1.3 | — | — | 2.3 | — | 0.7 |
| Crushing strength (N) | 5 | 200 | 6 | 183 | 0 | 200 | 79 | 3 | 103 | 9 |
| Disintegration time | 172 | — | 146 | — | 128 | — | — | 147 | — | 125 |
| (min) | 56 | | 60 | | 58 | | | 50 | | >90 |

Procedure

(i) The material of step (iv) was mixed with the remaining quantity of Magnesium stearate (external), Sodium starch glycolate (Primojel®)(external), Soda Ash IPH (external) or micro crystalline cellulose (external).

(ii) The bulk and tap density was measured and the Carr-index was calculated.

(iii) The material of step (vi) was compressed using a tablet compression machine at the indicated compression force.

(iv) The friability, crushing strength, and the disintegration time were measured.

### Example 4

### Comparative Dissolution Study for SLV306 Formulation with Soda Ash IPH and a Traditionally Formulated Tablet

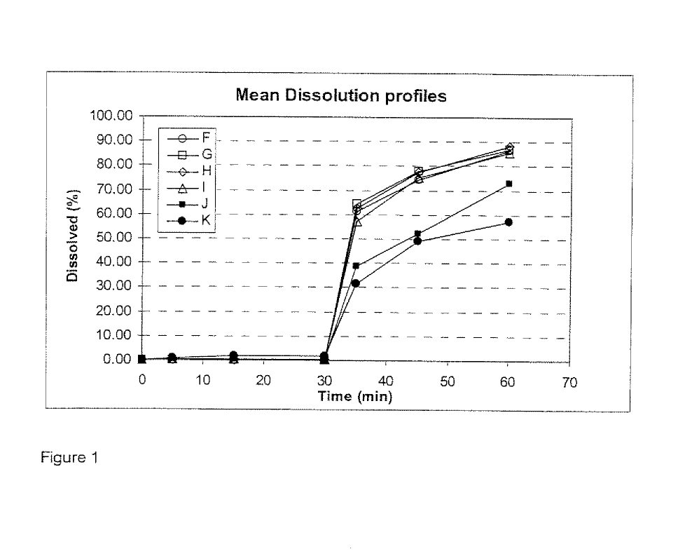

A comparative dissolution study according to the method described in Example 1 (method a) was carried out on one batch of a traditionally formulated tablet (Tablet K, prepared as described in Example 2) and five 600/965 batches of the calcium salt of SLV-306 (S—Ca) prepared according to Example 3 (see batch indications in table)(Tablet F compressed at 9 kN, Tablet G compressed at 12 kN, Tablet H compressed at 6 kN, tablet I compressed at 11 kN and tablet J compressed at 10 kN). The release profile of these formulations is given in the table below and depicted in FIG. 1 (◯=Formulation F; □=Formulation G; ⋄=Formulation H; Δ=Formulation I; ▪=Formulation J; ●=Formulation K)

Table Release of S—Ca from different formulations

| Time | F | G | H | I | J | K |
|---|---|---|---|---|---|---|
| (minutes) | (% rel) | (% rel) | (% rel) | (% rel) | (% rel) | (% rel) |
| 0 | 0.00 | 0.00 | 0.01 | 0.13 | 0.00 | 0.0 |
| 5 | 0.04 | 0.19 | 0.19 | 0.41 | 0.25 | 1.1 |
| 15 | 0.13 | 0.21 | 0.22 | 0.40 | 0.23 | 1.6 |
| 30 | 0.14 | 0.12 | 0.25 | 0.32 | 0.26 | 1.9 |
| 35 | 61.10 | 64.45 | 62.54 | 57.35 | 36.74 | 31.4 |
| 45 | 74.44 | 78.15 | 77.61 | 75.02 | 52.26 | 49.1 |
| 60 | 86.43 | 87.03 | 88.40 | 85.75 | 72.91 | 57.4 |

From this study it was concluded that a formulation of S-Ca with a high drug load, having a reasonable size and a favourable release profile was prepared.

I claim:

1. An oral pharmaceutical composition of an active compound of the formula (I): 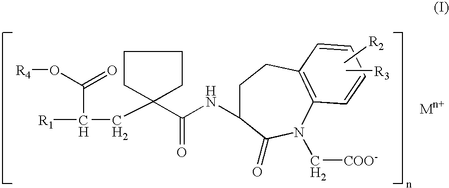
wherein:
R₁ is chosen from:
(1) (C₁-C₆)alkoxy(C₁-C₆)alkyl, which is optionally substituted by a (C₁-C₆)alkoxy;
(2) phenyl-(C₁-C₆)-alkyl and phenyloxy-(C₁-C₆)alkyl, wherein the phenyl group is optionally substituted with (C₁-C₆)-alkyl, (C₁-C₆)alkoxy or halogen; and
(3) naphtyl-(C₁-C₆)-alkyl;
R₂ and R₃ are both independently hydrogen or halogen;
R₄ is a biolabile ester forming group;
M is a hydrogen or a metal ion; and
n is 1, 2 or 3;
said composition comprising
a) said active compound in an amount ranging from 10 to 65% of the total weight of the formulation;
b) at least 10% w/w of an alkaline compound or a mixture of alkaline compounds; and
c) optionally, at least one auxiliary material in an amount ranging between 1 % and 45% of the total weight of the formulation; and wherein the composition does not contain a surfactant.

2. The oral pharmaceutical composition as claimed in claim 1, wherein the alkaline compound is chosen from inorganic and organic alkaline compounds.

3. An oral pharmaceutical composition of an active compound of the formula (I): 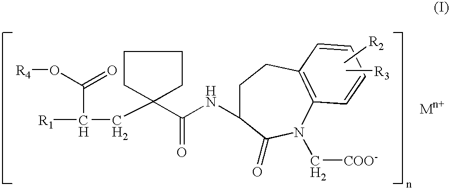
wherein:
R₁ is chosen from:
(1) (C₁-C₆)alkoxy(C₁-C₆)alkyl, which is optionally substituted by a (C₁-C₆)alkoxy;
(2) phenyl-(C₁-C₆)-alkyl and phenyloxy-(C₁-C₆)-alkyl, wherein the phenyl group is optionally substituted with (C₁-C₆)alkyl, (C₁-C₆)-alkoxy or halogen; and
(3) naphtyl-(C₁-C₆)-alkyl;
R₂ and R₃ are both independently hydrogen or halogen;
R₄ is a biolabile ester forming group;
M is a hydrogen or a metal ion; and
n is 1, 2 or 3;
said composition comprising
a) said active compound in an amount ranging from 10 to 65% of the total weight of the formulation;
b) at least 10% w/w of a sodium carbonate having a particle size distribution, wherein more than 97% of the particles are smaller than approximately 500 μm, more than 40% of the particles are smaller than approximately 160 μm, and more than 10% of the particles are smaller than approximately 63 μm; and
c) optionally comprising at least one auxiliary material in an amount ranging between 1% and 45% of the total weight of the formulation.

4. The oral pharmaceutical composition as claimed in claim 3, wherein said sodium carbonate has a particle size distribution wherein more than 98% of the particles are smaller than approximately 500 μm, more than 60% of the particles are smaller than approximately 160 μm, and more than 30% of the particles are smaller than approximately 63 μm.

5. The oral pharmaceutical composition as claimed in claim 4, wherein said sodium carbonate has a particle size distribution wherein approximately 99.8% of the particles is smaller than approximately 500 μm, approximately 80% of the particles is smaller than approximately 160 μm, and approximately 40% of the particles is smaller than approximately 63 μm.

6. The oral pharmaceutical composition as claimed in claim 3, wherein the sodium carbonate has a specific surface area of more than approximately 1.0 m²/g.

7. The oral pharmaceutical composition as claimed in claim 6, wherein said sodium carbonate has a specific surface area of more than approximately 1.5 m²/g.

8. The oral pharmaceutical composition as claimed in claim 7, wherein said sodium carbonate has a specific surface area of approximately 2.0 m²/g.

9. The oral pharmaceutical composition as claimed in claim 3, wherein said sodium carbonate is present in an amount of at least approximately 20% w/w of the composition.

10. The oral pharmaceutical composition as claimed in claim 1, wherein M is calcium in its 2+ form.

11. The oral pharmaceutical composition as claimed in claim 1, wherein the amount of alkaline compound is more than approximately 55% wlw.

12. The oral pharmaceutical composition as claimed in claim 11, wherein the amount of alkaline compound is approximately 60% w/w.

13. The oral pharmaceutical composition as claimed in claim 1, wherein said active compound is the calcium salt of 1H-1-Benzazepine-1-acetic acid.

14. The oral pharmaceutical composition as claimed in claim 1, wherein the composition is in the form of granules, compressed tablets, or capsules.

15. A method of preparing an oral pharmaceutical composition as claimed in claim 1, comprising:
a) mixing the active compound of formula (I) with one or more alkaline compounds and, optionally, with one or more auxiliary materials;
b) compacting the mixture;
c) milling and sieving the granules obtained from said compacting, and optionally, mixing said sieved granules with one or more auxiliary materials; and
d) optionally compressing the mixture into tablets, optionally followed by coating, and/or optionally filling the mixture into capsules.

16. A method of preparing an oral pharmaceutical composition as claimed in claim 1, comprising:
a) mixing the active compound with one or more auxiliary materials;
b) granulating said mixture with an organic solvent;
c) removing the organic solvent to obtain granules;
d) milling and sieving the granules, and mixing the sieved granules with the remaining portion of the auxiliary materials; and
e) optionally, compressing the mixture into tablets, optionally followed by coating, and/or optionally filling the mixture into capsules.

17. The oral pharmaceutical composition as claimed in claim 1, wherein the composition has a dissolution of at least approximately 50% within approximately 5 minutes as measured using the USP apparatus 2 configuration at a paddle speed of 50 rpm at 37.0° C. and at pH 6.8.

18. The oral pharmaceutical composition as claimed in claim 17, wherein the composition has a dissolution of at least approximately 65% within approximately 15 minutes as measured using the USP apparatus 2 configuration at a paddle speed of 50 rpm at 37.0° C. and at pH 6.8.

19. The oral pharmaceutical composition as claimed in claim 18, wherein the composition has a dissolution of at least approximately 75% within approximately 30 minutes as measured using the USP apparatus 2 configuration at a paddle speed of 50 rpm at 370° C. and at pH 6.8.

20. The oral pharmaceutical composition as claimed in claim 1, wherein the composition has a dissolution of at least approximately 50% within approximately 5 minutes, approximately 65% within approximately 15 minutes and approximately 75% within approximately 30 minutes as measured using the USP apparatus 2 configuration at a paddle speed of 50 rpm at 37.0° C. and at pH 6.8.

21. The oral pharmaceutical composition as claimed in claim 17, wherein the composition has a dissolution of less than approximately 5% within approximately 30 minutes as measured using the USP apparatus 2 configuration at a paddle speed of 50 rpm at approximately 37.0° C. and at pH 2.0

22. The oral pharmaceutical composition as claimed in claim 1, wherein M is a bivalent metal ion.

23. The oral pharmaceutical composition as claimed in claim 2, wherein the organic alkaline compounds are chosen from sodium bicarbonate, potassium bicarbonate, sodium carbonate, potassium carbonate, sodium citrate, tris buffer, triethanolamine, alkaline hydroxides, alkaline phosphates, and mixtures of any one or more of said alkaline compounds

24. The oral pharmaceutical composition as claimed in claim 3, wherein M is a bivalent ion.

25. The oral pharmaceutical composition as claimed in claim 12, wherein said active compound is the calcium salt of 1H-1-Benzazepine-1-acetic acid and is in its 3S,2′R form.

26. The oral composition as claimed in claim 23, wherein the alkaline hydroxides are chosen from sodium hydroxide, potassium hydroxide and magnesium hydroxide.

27. The oral composition as claimed in claim 23, wherein the alkaline phosphates are chosen from dipotassium hydrogen phosphate and meglumine.

28. The oral pharmaceutical composition as claimed in claim 1, wherein the composition improves gastrointestinal blood flow.

29. The oral pharmaceutical composition as claimed in claim 1, wherein the composition improves hypertension.

30. The oral pharmaceutical composition as claimed in claim 1, wherein the composition improves or protects against or both improves and protects against cardiac damages induced by at least one anti-cancer drug.

31. The oral pharmaceutical composition as claimed in claim 30, wherein the anti-cancer drug is adriamycin.
